(19) United States
(12) Patent Application Publication
(10) Pub. No.: US 2019/0145800 A1
(43) Pub. Date: May 16, 2019

(54) DISPLACEMENT MEASUREMENT APPARATUS

(71) Applicant: Mitutoyo Corporation, Kawasaki (JP)

(72) Inventor: Toru Yaku, Kanagawa (JP)

(21) Appl. No.: 16/178,027

(22) Filed: Nov. 1, 2018

(30) Foreign Application Priority Data
Nov. 10, 2017 (JP) 2017-217250

(51) Int. Cl. G01D 5/347; G01N 33/00

(52) U.S. Cl. CPC G01D 5/34746; G01N 33/0044

(57) ABSTRACT

To enable a displacement measurement apparatus to detect its own environment and discover, in advance, a worsening in the environment or a cause of a drop in measurement precision. The displacement measurement apparatus includes a scale contained within a scale housing, and a slider capable of moving relative to the scale, the slider measuring displacement relative to the scale. Furthermore, at least one of the slider and the scale housing is provided with a second sensor. The second sensor detects at least one of hydrogen sulfide, sulfur dioxide, nitrogen oxides, oxygen, vibration, sound, and magnetism.

### TECHNICAL FIELD

The present invention relates to a displacement measurement apparatus.

### BACKGROUND ART

Displacement measurement apparatuses, i.e., encoders, are used for precise positional control in various types of industrial machinery (Patent Documents 1 and 2). Displacement measurement apparatuses include not only linear encoders, which measure linear displacement, but also rotary encoders, which measure rotation angles (or rotation amounts). The displacement measurement apparatus is mounted on a machine tool such as a milling machine or a lathe, and operations of the machine tool are controlled based on measurements taken by the displacement measurement apparatus.

### CITATION LIST

### Patent Literature

Patent Document 1: JP 2004-301541 A

Patent Document 2: JP 2017-067629 A

### SUMMARY OF INVENTION

### Technical Problem

A displacement measurement apparatus is used to control a machine tool, for example, but environments in which machine tools are used are often harsh. This can in some cases produce a rapid drop in measurement precision of a displacement measurement apparatus.

For example, a cutting fluid is used when processing a workpiece with a machine tool. Water-based cutting fluids are being used recently in order to reduce an impact on the environment. With a water-based cutting fluid, bacteria can grow in the fluid and produce hydrogen sulfide. Cutting fluids are managed appropriately and discarded periodically according to the operation guidelines of the managing factories, and workers of course manage cutting fluids appropriately according to the guidelines.

Structurally speaking, a displacement measurement apparatus has openings in housings containing scales, stators, and the like. A fluid can enter through these openings and accumulate in the interior of the apparatus. While such openings are of course designed to be as fluid-tight as possible, it is not possible to completely prevent fluid entry. If a cutting fluid that has accumulated in the apparatus interior produces hydrogen sulfide, the hydrogen sulfide may corrode the electrodes of electronic components installed in the displacement measurement apparatus. This in turn can lead to contact problems with connectors, relays, and the like, for example.

Optical displacement measurement apparatuses, meanwhile, include optical devices having reflective films (e.g., a reflective diffraction grating), and hydrogen sulfide can corrode such reflective films and reduce the reflectivity.

The housings containing scales, stators, and the like have fluid-tight structures, which is particularly problematic in that hydrogen sulfide produced by bacteria growing in the housings can quickly become highly concentrated.

Mobile parts of a machine tool are provided with bearings such as ball bearings. Fine vibrations arise in the machine tool when the bearings wear down, and these vibrations can have a major effect on the precision of a displacement measurement apparatus.

Furthermore, linear motors are sometimes used as driving sources in machine tools. A high level of magnetic leakage from a linear motor will adversely affect the precision of a displacement measurement apparatus.

Incidentally, it can be difficult to ascertain the cause of a drop in the precision of a displacement measurement apparatus even if the apparatus is taken apart. For example, a cutting fluid that has entered a housing may evaporate after hydrogen sulfide has been produced. Vibrations, magnetic leakage, and the like are problems that only arise while a machine tool is operating, and thus a careful examination of the displacement measurement apparatus alone will not reveal the cause of reduced precision. A repair worker may discover the cause by visiting the site and inspecting the installation conditions, but doing so is labor-intensive.

An object of the invention is to enable a displacement measurement apparatus to detect its own environment and discover, in advance, a worsening in the environment or a cause of a drop in measurement precision.

### Solution to Problem

A displacement measurement apparatus according to an aspect of the invention includes a scale contained within a scale housing, and a slider configured to move relative to the scale and measure displacement relative to the scale. At least one of the slider and the scale housing is provided with a second sensor, the second sensor being configured to detect at least one of hydrogen sulfide, sulfur dioxide, nitrogen oxides, oxygen, vibration, sound, and magnetism.

According to an aspect of the invention, preferably, when a position data request signal is received from an external device, the displacement measurement apparatus obtains a measurement value from the second sensor along with position data, and

when data is sent from the displacement measurement apparatus to the external device, the measurement value from the second sensor is sent along with the position data.

According to an aspect of the invention, preferably, the displacement measurement apparatus further includes a warning device, and in a case where the measurement value from the second sensor has reached a predetermined threshold, a warning is provided to a user from the warning device.

### BRIEF DESCRIPTION OF DRAWINGS

FIG. 1 is a diagram illustrating a configuration of a displacement measurement apparatus.

FIG. 2 is an exploded perspective view of a displacement measurement apparatus.

FIG. 3 is a diagram schematically illustrating a cross-sectional view of a displacement measurement apparatus.

### DESCRIPTION OF EMBODIMENTS

An embodiment of the invention will now be described with reference to the drawings and the reference numerals appended to the elements illustrated in the drawings.

### First Embodiment

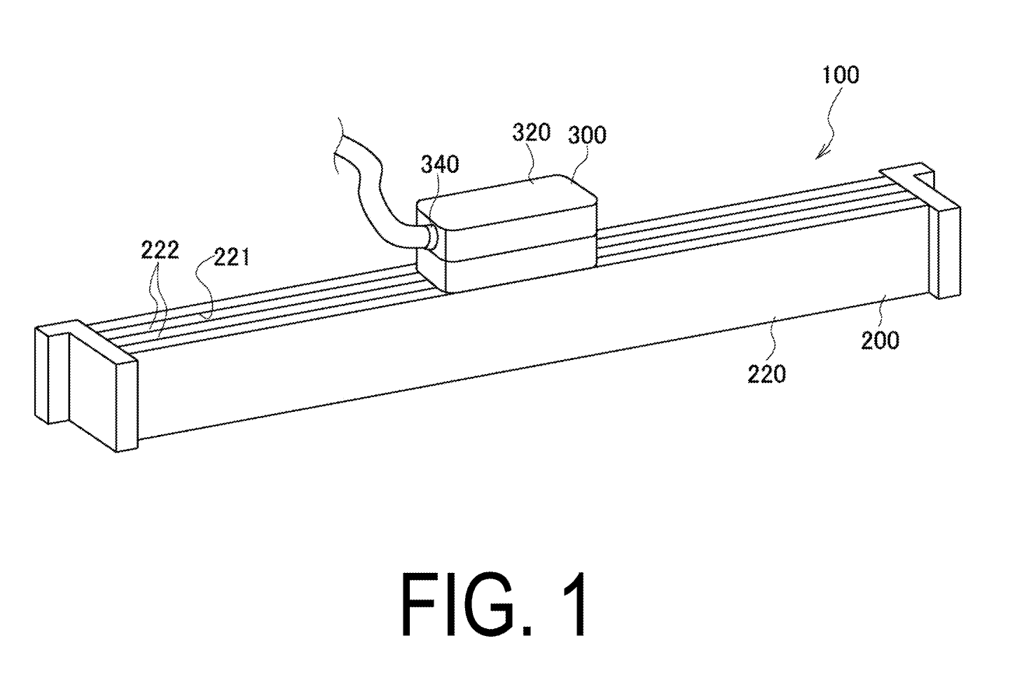

FIG. 1 is a diagram illustrating a configuration of a displacement measurement apparatus 100.

Although the displacement measurement apparatus 100 itself is already known, it will be described briefly here.

The displacement measurement apparatus 100 includes a longitudinal scale unit 200, and a slider 300 capable of sliding relative to the scale unit 200.

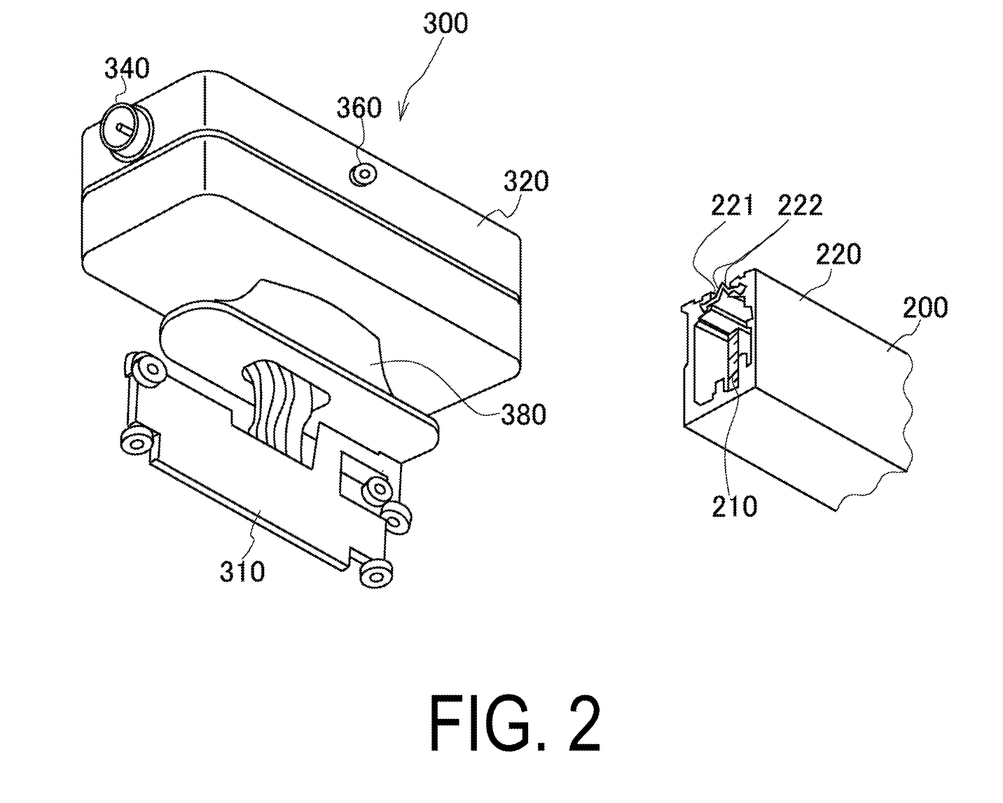

FIG. 2 is an exploded perspective view of the displacement measurement apparatus 100.

The scale unit 200 includes a longitudinal main scale 210 and a scale housing 220 containing the main scale 210.

The main scale 210 is constituted primarily of a glass substrate, and marks are formed along a length measurement axial direction. When the apparatus is a photoelectric type, the marks are a diffraction grating.

The scale housing 220 has a long, hollow shape and is often made from a (lightweight) metal such as aluminum.

The scale housing 220 includes a slit 221 in a side face running along the axial direction. The main scale 210 is attached and fixed to the inside of the scale housing 220.

Thin plates 222 and 222 made from a resin (rubber) are provided on both side walls of the slit 221 to close off the opening of the slit 221. These two rubber plates 222 and 222 ensure, as much as possible, that liquid (oil, cutting fluid, and the like) do not enter into the scale housing 220 from the slit 221.

The slider 300 is capable of moving relative to the scale housing 220 in a longitudinal direction, and detects a relative amount of displacement or relative position with respect to the main scale 210.

The slider 300 includes a traveling member 310 that travels along the main scale 210 so as to follow the main scale 210, a carriage part 320 that is located outside the scale housing 220 and slides along the scale unit 200, and a neck part 380, serving as connecting means, that connects the traveling member 310 and the carriage part 320. A detection unit is installed in the traveling member 310, and the detection unit detects a relative amount of displacement with respect to the main scale 210. The detection unit is constituted by a light-emitting element and a light-receiving element, for example.

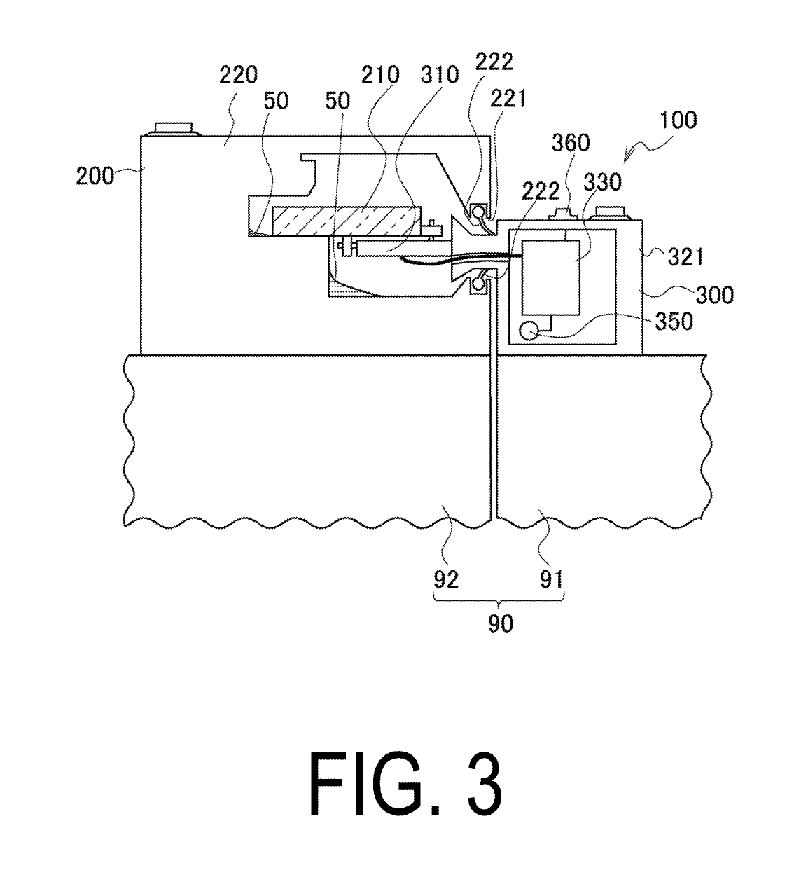

FIG. 3 is a diagram schematically illustrating a cross-sectional view of the displacement measurement apparatus 100.

(In FIG. 3, cross-section hatching has been omitted for the sake of clarity.)

In FIG. 3, the displacement measurement apparatus 100 is attached to a mobile part of a machine tool.

Specifically, the carriage part 320 is attached to a stage 91 side of a mobile stage 90, and the scale unit 200 is attached to a base 92 side of the mobile stage 90.

The carriage part 320 includes a carriage housing 321 and an electrical unit 330 contained within the carriage housing 321. A light emission control circuit of a light source, a signal processing unit that processes light-reception signals from the light-receiving element, or the like is provided as the electrical unit 330. The carriage housing 321 is also provided with a connector 340 for connecting to an external device.

Furthermore, a sensor (a second sensor) 350 is disposed within the carriage housing 321.

The sensor 350 is a hydrogen sulfide sensor 350 that detects hydrogen sulfide. The hydrogen sulfide sensor 350 is electrically connected to the electrical unit 330, and a detection signal output from the hydrogen sulfide sensor 350 is output to the exterior through the electrical unit 330 and the connector 340.

For the displacement measurement apparatus, a position detection sensor (e.g., the light-receiving element) serves as a first sensor, and thus the hydrogen sulfide sensor is the second sensor.

Note that FIG. 3 illustrates a state in which a cutting fluid 50 has entered and accumulated within the scale housing 220.

With a configuration such as that described in the present embodiment, bacteria may grow in the cutting fluid that has entered and accumulated within the scale housing, and the bacteria may then produce hydrogen sulfide. The hydrogen sulfide can fill the interior of the scale housing, enter into the carriage housing 321 through signal line paths, and can of course escape through the slit 221 to the exterior.

The hydrogen sulfide is detected by the hydrogen sulfide sensor 350.

Hydrogen sulfide concentration data measured by the hydrogen sulfide sensor 350 is sent to an external device (not shown) through the electrical unit 330.

For example, when the displacement measurement apparatus 100 has received a position data request signal from the external device, the electrical unit 330 obtains the hydrogen sulfide concentration data along with position data.

When sending data to the external device from the displacement measurement apparatus 100, the hydrogen sulfide concentration data is sent after the position data. As a result, the external device obtains the concentration of hydrogen sulfide at a place where the displacement measurement apparatus 100 is installed, along with the position data. If there is a high hydrogen sulfide concentration, the external device issues a warning to a user to prompt the user to purify the environment.

Furthermore, because the hydrogen sulfide concentration data is sent along with the position data, a region in a movement range of the carriage part 320 where the hydrogen sulfide concentration is particularly high can be identified when the carriage part 320 scans the movement range.

The scale 210 can be as long as 2 m to 3 m. Building the hydrogen sulfide sensor 350 into the carriage part 320 of the displacement measurement apparatus 100 makes it possible to obtain a detailed hydrogen sulfide concentration map over the broad 2-3 m range. This makes it possible to selectively purify high hydrogen sulfide concentration ranges, which makes the environment purification more efficient.

### MODIFIED EXAMPLE 1

A warning lamp 360 may be attached to the carriage part 320, and the warning lamp 360 may be lit up to communicate the status of the hydrogen sulfide concentration measured by the hydrogen sulfide sensor 350.

The status of the hydrogen sulfide concentration is set as follows, for example (the thresholds are merely examples, and the set numbers or values of the thresholds can be changed as desired).

Hydrogen sulfide concentration of less than 1 ppm: safe

Hydrogen sulfide concentration of 1 ppm or more but less than 5 ppm: caution

Hydrogen sulfide concentration of 5 ppm or more: danger

The light-up pattern of the warning lamp 360 is varied depending on the status. For example, the warning lamp 360 is green during the “safe” status, yellow during the “caution” status, and red during the “danger” status.

This makes it possible to elicit caution on the user.

Status information can also be sent to the external device at this time.

The warning lamp 360 may be replaced with a warning speaker that emits a warning sound.

### MODIFIED EXAMPLE 2

The foregoing embodiment describes an example in which the hydrogen sulfide sensor 350 is built into the carriage part 320.

This configuration is advantageous because the carriage part 320 already contains the electrical unit 330, and it is therefore easy to electrically connect the hydrogen sulfide sensor 350 to the electrical unit 330.

However, the hydrogen sulfide sensor 350 may be disposed within the scale housing 220.

It is conceivable that the cutting fluid 50 will accumulate within the scale housing 220 and produce hydrogen sulfide, and thus it is also reasonable to dispose the hydrogen sulfide sensor 350 within the scale housing 220.

As means for exchanging electrical signals, a signal line from the scale housing 220 may be laid separately, or signals may be sent and received wirelessly by the hydrogen sulfide sensor 350.

The displacement measurement apparatus 100 will not necessarily always be installed horizontally, and may in extreme cases be installed with the scale 210 oriented vertically to measure height displacement. Alternatively, if the mobile part of the machine tool is tilted from the outset, the scale 210 may be attached in a tilted state.

In such a case, with hydrogen sulfide being slightly heavier than air, the hydrogen sulfide will fill the scale housing 220 from the bottom with respect to the direction of gravity, and it is thus preferable that the hydrogen sulfide sensor 350 be positioned as low as possible within the scale housing 220.

### MODIFIED EXAMPLE 3

Although the sensor 350 is described as the hydrogen sulfide sensor 350 in the foregoing embodiment, the sensor 350 installed in the displacement measurement apparatus 100 may be a vibration sensor, a microphone, or a magnetism sensor.

The vibration sensor may be an accelerometer.

If there is an indication of some kind of malfunction in the mobile part to which the displacement measurement apparatus 100 is attached (e.g., the mobile stage 90 of the machine tool), it may appear in the displacement measurement apparatus 100 as minute vibrations. These vibrations are detected by the vibration sensor of the displacement measurement apparatus 100.

Alternatively, if there is an indication of some kind of malfunction in the mobile part (e.g., the mobile stage 90 of the machine tool), components may rub together, creak, and the like and produce faint noise. This faint noise travels to the displacement measurement apparatus 100 and is therefore detected.

When a linear motor is used as the driving source of the mobile part (e.g., the mobile stage 90 of the machine tool), magnetic leakage can occur due to component degradation and the like. Such magnetic leakage can affect the operations of the displacement measurement apparatus 100. When the displacement measurement apparatus 100 is a magnetic linear encoder, in which a magnetic pattern is provided in the scale 210, magnetism infiltrating from the exterior will directly affect the reading of the scale.

Vibration data, sound data, or magnetism data detected by the sensor is sent to the external device from the displacement measurement apparatus 100 along with the position data.

Accordingly, a region in the movement range of the carriage part 320 where high levels of vibrations, sounds, or magnetism are marked can be identified during scanning of the movement range. A range where high levels of vibrations, sounds, or magnetism are marked can thus be examined in detail, which enables the early detection of indications that the machine tool is malfunctioning.

The invention is not intended to be limited to the above-described embodiment, and appropriate variations can be made thereon without departing from the essential spirit of the invention.

Although a hydrogen sulfide sensor that detects hydrogen sulfide is given as an example of the sensor 350, the sensor may be configured to detect sulfur dioxide, nitrogen oxides, or the like. Air in industrial zones typically contains a higher concentration of sulfur dioxide, nitrogen oxides, and the like than normal. Such compounds may build up within the scale housing and permeate into the water-based cutting fluid, producing sulfuric acid or nitric acid and affecting the scale, electrical components, and the like.

The type of gas to be detected by the sensor 350 is not limited to the gases listed above, and may be any gas that affects the human body or electronic devices (displacement measurement apparatuses, machine tools, and the like). For example, too low or too high an oxygen concentration affects the human body, and thus in some cases an oxygen sensor may be used as the sensor 350.

The same of course applies to physical amounts such as vibrations, sounds, or magnetism, and the sensor 350 may be configured to detect any type of physical amount that affects the human body or electronic devices (displacement measurement apparatuses, machine tools, and the like).

Although the foregoing embodiment describes a linear displacement measurement apparatus that measures linear displacement using a linear scale as an example, the measurement apparatus may of course be a rotary encoder.

A plurality of the sensors 350 may be provided instead of a single sensor, and the plurality of sensors may be the same type or different types.

For example, the displacement measurement apparatus may be provided with two or more sensors selected from among a hydrogen sulfide sensor, a vibration sensor, a microphone, a magnetism sensor, a sulfur dioxide sensor, and a nitrogen oxide sensor, depending on what is to be detected.

Furthermore, two or more of the same type of sensor may be used, e.g., providing three hydrogen sulfide sensors, one on each side of the long scale and one in the middle.

### REFERENCE SIGNS LIST

50 Cutting fluid
90 Mobile stage
91 Stage
92 Base
100 Displacement measurement apparatus
200 Scale unit
210 Main scale
220 Scale housing
221 Slit
222 Rubber thin plate
300 Slider
310 Traveling member
320 Carriage part
321 Carriage housing
330 Electrical unit
340 Connector
350 Sensor
360 Warning lamp
380 Neck part

1. A displacement measurement apparatus comprising:
a scale; and
a slider configured to move relative to the scale and measure displacement relative to the scale,
wherein at least one of the slider and the scale is provided with a second sensor, the second sensor being configured to detect at least one of hydrogen sulfide, sulfur dioxide, nitrogen oxides, oxygen, vibration, sound, and magnetism.

2. The displacement measurement apparatus according to claim 1, wherein
when a position data request signal is received from an external device, the displacement measurement apparatus obtains a measurement value from the second sensor along with position data; and
when data is sent from the displacement measurement apparatus to the external device, the measurement value from the second sensor is sent along with the position data.

3. The displacement measurement apparatus according to claim 1, further comprising:
a warning device,
wherein in a case where the measurement value from the second sensor has reached a predetermined threshold, a warning is provided to a user from the warning device.

4. The displacement measurement apparatus according to claim 2, further comprising:
a warning device,
wherein in a case where the measurement value from the second sensor has reached a predetermined threshold, a warning is provided to a user from the warning device.

5. The displacement measurement apparatus according to claim 1, further comprising a scale housing,
wherein the scale is contained within the scale housing.
